(19) United States
(12) Patent Application Publication
(10) Pub. No.: US 2011/0122486 A1
(43) Pub. Date: May 26, 2011

(54) Plasma-Deposited Electrically Insulating, Diffusion-Resistant and Elastic Layer System

(75) Inventors: Heinz Werner Busch, Auleweg 2 (DE); Udo Heinrich Grabowy, Euskirchen-Flamersheim (DE); Lisa Kleinen, Koln (DE)

(73) Assignee: TECHNISCHE UNIVERSITÄT KAISERSLAUTERN, Kaiserslautern (DE)

(21) Appl. No.: 12/528,320

(22) PCT Filed: Feb. 22, 2008
(86) PCT No.: PCT/EP08/01388

§ 371 Date: Dec. 28, 2010

(30) Foreign Application Priority Data
Feb. 23, 2007 (DE) 10 2007 008 861.4

(51) Int. Cl. G02B 1/12; B32B 9/04; H01L 51/52; A61N 1/375; C23C 16/513; G02B 5/20

(52) U.S. Cl. 359/359; 428/411.1; 428/408; 428/702; 428/446; 427/569

(57) ABSTRACT

A multilayer system on a substrate, the multilayer system being applied to the substrate by plasma deposition, characterized in that the multilayer system is configured such that it has substantial diffusion resistance to ions in an aqueous solution, wherein the current produced by the diffusion of the ions with the connection of an electric field gradient of more than 10⁴V/m, preferably more than 10⁵V/m, most preferred more than 10⁷V/m is I_{Ion}<6.5×10⁻⁸ A/cm², preferably I_{Ion}<6.5×10⁻¹⁰ A/cm², particularly I_{Ion}<1×10⁻¹² A/cm².

### FIELD OF THE INVENTION

The invention relates to a multilayer system on a substrate, in particular a multilayer system which is applied to the substrate with the aid of plasma deposition. Furthermore, the invention relates to the use of a multilayer system of this type.

### PRIOR ART

Diffusion barriers are of very great interest for a broad range of applications. The number of developments and publications in this field is correspondingly large. Most relate to the diffusion suppression of oxygen and moisture, for example on PET beverage bottles or organic light emitting diode (OLED) systems. The migration of charge carriers in solid layer systems is particularly important in the semiconductor industry and electronics, where layers are accordingly often used as diffusion barriers (albeit in a dry environment) between individual functional layers, for example of a transistor.

Diffusion barriers based on silicon dioxide, which under dry conditions has a resistance of >10¹⁶Ω, are known in the art for reducing the diffusion of gas through PET beverage bottles. Layer systems of this type are not so well suited to use in an aqueous solution, in particular for suppressing an ion current in the potential gradient, as the resistance of SiO₂ decreases under moisture as a result of incipient hydrolysis processes. Metal ions, in particular alkali ions, such as for example the Na⁺ ions which are always present in body fluids, can diffuse through the gaps present in the SiO₂ network. The silicon nitride layers (for example Si₃N₄), which are likewise used in semiconductor structures, also tend to be unsuitable as diffusion barriers on electrodes in an aqueous solution. At the interface to the aqueous medium there is formed a hydrated layer (SiNₓO_{y}H_{z}) which permits the diffusion of protons (H⁺) into the layer system—an effect which makes these layers ideal for use in ISFETs (ion-sensitive field effect transistors) for determining pH.

US 2006/0208634 A1 describes plasma-deposited diffusion barriers based on a gradient layer having an organic and inorganic component, the inorganic component consisting substantially of silicon oxynitride (SiNₓO_{y}H_{z}) and the organic component consisting of plasma-polymerised films. The application describes the gradual stoichiometric gradation when changing from the organic to the inorganic component and presents the gradient layer as part of an organic light emitting diode (OLED) for providing protection from moisture and oxygen. For the silicon oxynitrides, the above-cited considerations apply concerning their suitability as diffusion barriers in aqueous electrolyte solutions on application of an electrical potential.

US 2007/0020451 A1 describes a barrier layer for protecting organic light emitting diodes from moisture and oxygen. In the barrier proposed in US 2007/0020451 A1, amorphous carbon layers and layers of “diamond-like glass” (DLG, an amorphous mixture of glass having various contents of embedded carbon, hydrogen, silicon, oxygen, fluorine, sulphur, titanium and copper) alternate with polymer layers. The layer system has, owing to the DLG layers, higher tolerance to expansion and forms, under the same expansion, fewer cracks than a sputtered SiOₓ layer.

Multilayered layer systems in which the individual layers have different moduli of elasticity have also already been described. Thus, U.S. Pat. No. 6,491,798 B2 proposes a laminated layer system for protecting a magnetic hard disk. In said document, thin layers having a high and low modulus of elasticity are successively sputtered (DC magnetron method) by a graphite target having various process gas compositions (argon, hydrogen, nitrogen in a ratio of 6:1:3 or 6:4) at various process pressures (9×10⁻³ mbar or 1.3×10⁻² mbar). The laminate is intended to protect the hard disk from impacts of the write head in that it has a modulus of elasticity (at least 150 GPa) which is at least twice as high in the direction perpendicular to the surface as parallel thereto.

The laminate described in U.S. Pat. No. 6,491,798 B2, which is made up of sputtered carbon layers, does not have sufficient diffusion resistance.

Barrier layers for the purpose of restricting the diffusion of ions in aqueous or other solutions are known, for example, from U.S. Pat. No. 6,891,155. U.S. Pat. No. 6,891,155 discloses a dielectric film which maintains its diffusion resistance to ions and moisture even in an aqueous solution when electrical potential is applied. The film consists of a base layer and a capping layer, wherein the capping layer can consist of various types of layer such as, for example, silicon oxynitrides of various stoichiometric composition, all conventional dielectrics (nitridic, oxidic, ceramic) and also of diamond-like carbon. The layer system is intended to be used in microfluidic systems in what are known as lap-on-a-chip applications. Drawbacks of the layer system according to U.S. Pat. No. 6,891,155 include the high layer thickness while having at the same time a small number of layers; this results in the formation of cracks under loading and expansion. In particular, this system is not suitable for use on flexible substrates.

U.S. Pat. No. 5,769,874 describes a diffusion-resistant coating which is a hermetic encapsulation for a medical implant, the encapsulation being carried out around a metallic housing made of titanium.

The restriction of the diffusion of oxygen or water vapour by applying a polymer layer, in particular made of parylene, or an inorganic layer, for example SiOₓN_{y}, in combination with a polymer layer, in particular parylene, including for uses in an aqueous solution and biological medium, are described, for example, by Andrew Spence, Keith Neeves, Devon Murphy et al., Flexible multielectrode can resolve multiple muscles in an insect appendage, Journal of Neuroscience Methods 159 (2007) 116, J. H. Lee, C. H. Jeong, H. B. Kim et al., Characteristics of SiOxNy films deposited by inductively coupled chemical vapor deposition using HMDS/NH₃/O₂/Ar for water vapor diffusion barrier, Thin Solid Films 515 (2006) 917 or C. C. Chiang, D. S. Wuu, H. B. Lin et al., Deposition and permeation of SiNx/Parylene multilayers on polymeric substrates, Surface and Coatings Technology 200 (2006) 5843.

D. Feili, M. Schuettler, T. Doerge et al., Encapsulation of organic field effect transistors for flexible biomedical microimplants, Sensors and Actuators A 120 (2005) 101 describes a coating comprising parylene for flexible components and implants. Feili for use on field effect transistors for flexible biomedical implants. The encapsulation of a retina implant has become known from Damien Rodger, James Weiland, Mark Humayun et al., Scalable high lead-count parylene package for retinal prostheses, Sensors and Actuators B 117 (206) 107.

Amorphous carbon layers can be deposited by a large number of plasma-technological methods. Reference is made in this regard to WO 03/035928. The depositing method described in WO 03/035928 allows particular deposition conditions and is ideal for producing the diffusion-resistant core layers and carbon-based, resilient matrix layers.

Satisfactory diffusion resistance cannot be attained under biological conditions by way of a simple amorphous carbon layer.

### DESCRIPTION OF THE INVENTION

The object of the invention is to overcome the drawbacks of the prior art.

According to the invention, the object is achieved by a multilayer system on a substrate, the multilayer system being applied to the substrate with the aid of plasma deposition. The multilayer system is constructed in such a way that it has substantial diffusion resistance to ions, the current produced by the diffusion of the ions in an aqueous solution on application of an electric field gradient of more than 10⁴ V/m, preferably more than 10⁵ V/m, most preferably more than 10⁷ V/m, being Iᵢₒₙ<6.5×10⁻⁸ A/cm², preferably Iᵢₒₙ<6.5×10⁻¹⁰ A/cm², in particular Iᵢₒₙ<1×10⁻¹² A/cm². Said field gradient is applied between the layer-remote side of the substrate and an electrode positioned at a distance of 1 mm before the surface of the layer system which is wetted with isotonic saline solution. The measurement was carried out using a picoamperemeter at a wetted area of 10 cm².

In a development of the invention, the multilayer system is distinguished in that it is constructed in such a way that it has substantial diffusion resistance to gases and/or water vapour and/or solvent vapours, the flow of water vapour through the layer system being Φ_{H2O}<3.7×10⁻⁸ mbar×l/(s×cm²), preferably Φ_{H2O}<3.7×10⁻⁹ mbar×l/(s×cm²), in particular Φ_{H2O}<3.7×10⁻¹⁰ mbar×l/(s×cm²), and the gas flow being Φ_{gas}<1.5×10⁻⁷ mbar×l/(s×cm²), preferably Φ_{gas}<1.5×10⁻⁹ mbar×l/(s×cm²), in particular Φ_{gas}<1.5×10⁻¹¹ mbar×l/(s×cm²). For measuring the flow, a gas and water vapour-permeable polymer sheet is coated with the multilayer system and clamped between two vacuum recipients. Water vapour up to a pressure of 1 bar or gas up to a pressure of 3 bar is introduced on one side (the high-pressure side). On the other (low-pressure) side (evacuated to 1×10⁻⁷ mbar by vacuum pumping), the respective flow through the sheet is determined with the aid of a quadrupole mass spectrometer.

In particular, the multilayer system is constructed in such a way that the layer system is substantially chemically stable in relation to acids and lyes in a pH range between 3 and 10, preferably between 2 and 12, in particular between 0 and 14. Advantageously, the multilayer system is constructed in such a way that substantial diffusion resistance is provided in the event of expansion in any desired direction parallel to the substrate of the multilayer system of less than 4%, in particular less than 10%, most preferably less than 25%.

In a preferred embodiment of the invention, the multilayer system is constructed in such a way that the multilayer system is thermally stable in the range from 0° C. to 121° C., in particular in the range from −10° C. to 150° C. and preferably in the range from −50° C. to 200° C. The term “thermally stable” refers in the present application to the fact that the multilayer system preserves its above-specified properties even during and after heating to the specified temperatures.

In particular, the multilayer system according to the invention has substantial diffusion resistance in an aqueous solution or in body fluid.

The multilayer system according to the invention, which is preferably a body-compatible layer system, can be deposited on different substrates in a single plasma deposition process by way of merging thin layers based on carbon and/or based on carbide and/or nitride and/or boride and/or metal oxide as an electrically insulating, diffusion-resistant barrier. The layer system preserves the aforementioned properties even under thermal, mechanical, chemical and electrical loading.

The multilayer system is an effective barrier against the diffusion of gases (such as for example helium, oxygen, air, water vapour, solvent vapours). It is impermeable to liquids (for example water, solvents, oils) and serves as a layer blocking ions and/or electrons even counter to a potential gradient (for example on application of an external voltage or counter to electrical boundary or double layers which build up).

The multilayer system is made up of components other than the layers or layer systems proposed in the above-cited documents. In particular, the multilayer system has, as a result of the novel arrangement of the individual components in the layer system, more extensive functionality than all the described systems. Preferably, the multilayer system on a substrate comprises at least one core layer (D), at least two matrix layers (E), at least one layer promoting adhesion to the substrate (GSB) and a capping layer of the multilayer system on the side (GBO) remote from the substrate, transition gradient layers (GED, GDE) being provided between the various layers, wherein the core layers (D) consist of amorphous carbon layers, a-C:H and/or ta-C:H and/or ta-C and/or DLCH and/or AlOₓ and/or SiOₓ and/or ZrOₓ and/or TaOₓ and/or TiOₓ and the resilient matrix layers (E) consist of a-C:H and/or PLCH and/or HC polymers and/or plasma-polymerised layers. Optionally, the multilayer system also comprises, in addition to these layers, an insulating layer A consisting of a-C:H and/or PLCH and/or a-C:H (soft) and/or a-C:H N and/or PLCHN and/or plasma-polymerised layers between the substrate and first resilient matrix layer close to the substrate. In this case, an adhesion promoter layer (GSA) is applied between the substrate and layer A, as is a gradient transition layer (GAB) between layer A and the first resilient matrix layer E close to the substrate in the multilayer system B.

In a preferred embodiment, the multilayer system comprises a number n of diffusion-resistant core layers (D) that is between 1 and 200, in particular between 3 and 9.

The number of resilient matrix layers E in a layer system of this type preferably assumes a value which is increased by one over the number of core layers, i.e. the number of resilient matrix layers is n+1 when n is the number of core layers. Preferably, the thickness of the diffusion-resistant core layers (D) is between 3 and 150 nm, preferably between 3 and 75 nm and in particular between 3 and 30 nm. The thickness of the resilient matrix layers (E) is preferably between 10 and 300 nm, preferably between 10 and 200 nm and in particular between 10 and 100 nm. In one particular embodiment, the diffusion-resistant core layers are distributed in the resilient matrix layers equidistantly or randomly or else with increasing density toward the centre of the layer system B. In the latter case, the thickness of the resilient matrix layers E at the centre of the multilayer system is thinner than at the edge.

Preferably, the thickness of the matrix layer (E) is 2 to 100 times, preferably 3 to 70 times the thickness of the core layers.

In a preferred embodiment of the invention, provision is made for the thickness of the matrix layer to be roughly (Y_{D}/Y_{E})^{0.5} times the thickness of the core layer. In this case, Y_{D} is the modulus of elasticity of the diffusion-resistant core layer and Y_{E} is the modulus of elasticity of the resilient matrix layer. This means that, for example if Y_{E}=40 GPa and Y_{D}=4 GPa, then it must be the case that d_{E}=3.16×d_{D}.

It is particularly preferred if the layer system further comprises a first transition gradient layer (GED) between elasticity layers (E) and core layers (D) and a second transition gradient layer (GDE) between the core layer (D) and elasticity layer (E).

The thickness of the two transition gradient layers (GED and GDE) is between 0.5 and 30 nm, preferably between 0.5 and 20 nm and in particular between 0.5 and 10 nm.

Advantageously, the transition gradient layers GSA and GSB respectively have in the multilayer system a coefficient of thermal expansion of between 10 and 150 ppm/K, preferably between 15 and 100 ppm/K, in particular between 15 and 70 ppm/K. The value ppm/K relates to a three-dimensional expansion.

Particularly preferably, the elasticity layers (E) have a modulus of elasticity of between 1 and 70 GPa, preferably between 3 and 50 GPa and in particular between 3 and 20 GPa.

In a developed embodiment, the multilayer system has as a substrate connection layer which is in contact with the substrate an insulating layer A consisting of a-C:H and/or DLCH and/or PLCH and/or HC polymers and/or a plasma-polymerised layer.

Furthermore, the system can have a substrate-remote capping layer (GBO, K, V), the substrate-remote capping layer comprising a-C:H and/or DLCH and/or ta-C:H and/or ta-C and/or PLCH.

Preferably, the substrate-remote capping layer has, at the substrate-remote surface, bond centres adapted to a connection system.

In particular, the bond centres comprise functional groups, in particular nitrogen-containing and/or oxygen-containing functional groups.

For example, the functional groups can comprise amino groups —NH2, carboxyl groups —COON, hydroxy groups —OH and/or foreign atoms and also generally anchor points for forming carbonyl and/or ester and/or ether bonds.

Preferably, the substrate is a polymer-like substrate and comprises parylene.

In a developed embodiment, the connection system comprises a polymer-like material, in particular parylene.

It is particularly preferred if the multilayer system described in the present document is reapplied to the connection system.

In particular, the multilayer system may be embodied as a UV filter and the transmission of electromagnetic radiation having wavelengths of between 200 and 400 nm may be less than 20%, preferably less than 10% and in particular less than 5%, the filter effect being independent of the substrate.

The multilayer systems according to the invention are distinguished by high body compatibility. In particular, human epithelial, endothelial and also blood cells and keratinocytes display no defensive reactions to the layer system.

The multilayer system according to the invention allows the restriction of diffusion of any desired particles and, in particular, of ions in an aqueous solution to be preserved, with simultaneously demonstrated body compatibility, on application of an electric field gradient, even under mechanical and thermal loading. As a result of the combination of basic diffusion resistance of the layer system with various “safety steps”, the layer system proposed in the present document withstands high loads and can remain fully operative for longer periods of time.

The multilayer system according to the invention displays the aforementioned properties in different environments, such as for example under vacuum, in air and in other gases, in solutions (aqueous or based on other solvents) and in particular in a biological medium, in plant and animal systems and most particularly in the human body, both in extracellular space, in organs, tissues and in physiological fluids (such as for example blood or lymph) but also in artificially cultivated tissues, cell cultures and fluids which simulate physiological fluids.

The invention further relates to a large number of devices provided with a multilayer system according to the invention. In this case, the encapsulations of the devices comprise, in particular, the multilayer systems.

Examples of these include electrically active medical implants, in particular implants for functional electrical stimulation (FES), implants with electrode systems for detecting bioelectric potential differences, for detecting neuronal innervation patterns, implants for electrical stimulation of nerve fibres, neuroprostheses, pacemakers, implants for dynamic myoplasty, implants for diaphragmatic or phrenic nerve stimulation with an encapsulation.

Further examples include electromechanical implants, in particular artificial hearts, VAD (ventricular-assisted device) systems, total artificial heart systems with an encapsulation, microimplants based on micromechanical systems with an encapsulation, electrodes and electronic circuits with an encapsulation to protect the electrodes and electric circuits, in particular, from the infiltration of moisture in environments having high air humidity, in aqueous solutions or other solvents, flat screens, in particular FPDs (flat panel displays) and LCDs (liquid crystal displays), and also organic light emitting diodes (OLEDs), for providing protection from diffusion and rapid ageing processes, microelectromechanical systems, in particular printer heads of inkjet printers, acceleration sensors for triggering airbags, packaging sheets and containers, in particular in the food sector for food products and beverages, in the pharmaceutical industry for medicaments and in the chemical industry for readily volatile solvents, petrols, corrosive liquids, hygroscopic solids and powders, storage containers for fuels, in particular petrol, hydrocarbons, hydrogen and highly volatile explosive mixtures, seals and covers and also fabrics and items of clothing.

In addition to the devices, the invention also includes the use of the multilayer system according to the invention in all the aforementioned devices.

### PREFERRED EXEMPLARY EMBODIMENTS OF THE INVENTION

The invention will be described hereinafter based on the exemplary embodiments and diagrams without limitation thereto.

In the drawings:

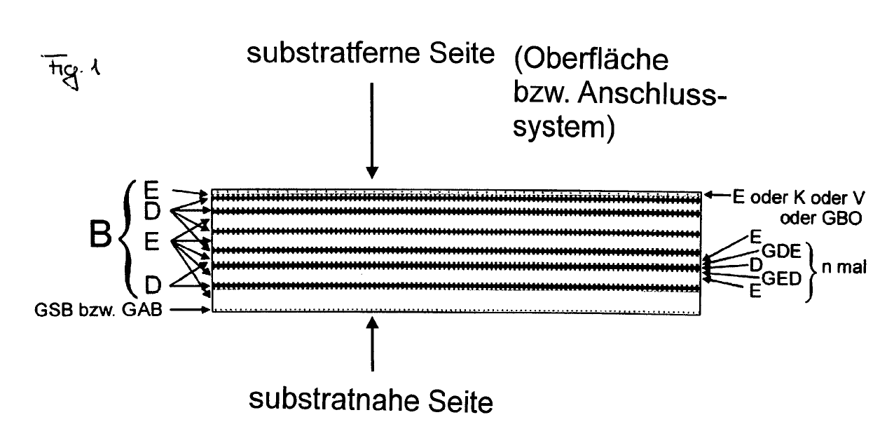

FIG. 1 shows the arrangement of the diffusion-resistant core layers (D) and the resilient matrix layers (E) and also the gradient layers (GED and GDE) in the layer system. The sequence E/GED/D/GDE/E is repeated in the layer system (B) n times. The layer system B is bonded either by the transition layer GSB to the substrate or by the transition layer GAB to a layer A deposited beforehand on the substrate in the same process. On the substrate-remote side (top), the system is capped by either a further resilient matrix layer E or an additional body-compatible capping layer K (in case E does not have sufficient body compatibility) or an additional protective layer V or a transition layer GBO which ensures permanent bonding of a further layer (or a layer system or an adhesive or a solid), for example by the provision of bond centres (for example of functional groups and/or foreign atoms) adapted to the connection system;

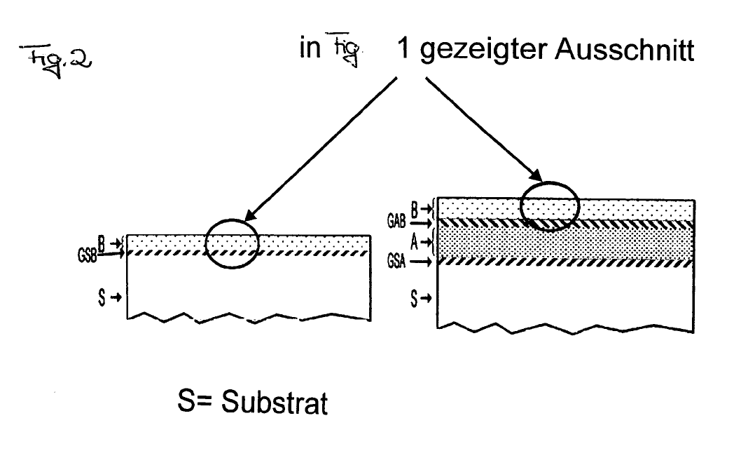

FIG. 2 shows layer system B (from FIG. 1) deposited on substrate S (left-hand side) or on a layer A deposited beforehand on the substrate S in the same process. The circles each indicate the detail illustrated in FIG. 1 (detailed view of layer system B);

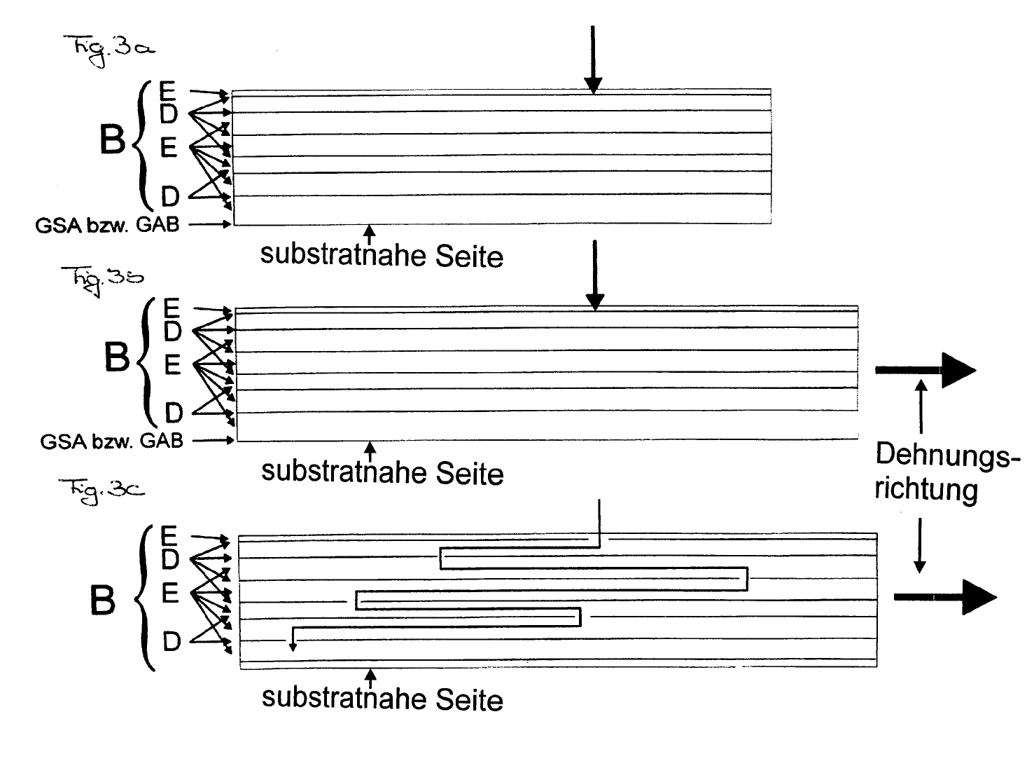

FIGS. 3a-c are schematic illustrations of the diffusion-resistant multilayer system. Black thin lines represent the diffusion-resistant core layers D, the white intermediate spaces represent the resilient matrix layers E. For the sake of clarity, the transition layers (GED and GDE) have not been shown (see in this regard FIG. 1); FIG. 3a shows layer system B as a diffusion barrier in the unloaded state. The direction of the arrow indicates the blocking direction; FIG. 3b shows layer system B as a diffusion barrier in the loaded state. In the event of mechanical and/or thermally induced expansion of the layer system, the diffusion-resistant core layers D remain intact. The loading is absorbed by the resilient matrix layers; FIG. 3c shows that, even under extreme loads, the diffusion resistance of the layer system B is preserved. Any microcracks in isolated diffusion-resistant core layers are compensated for by the large number of layers remaining intact. Damage to layers D lying directly one above another will be statistically distributed over the entire loaded length of the layer. The path of the species (gas, liquids, ions) to be held off by the layer system is in this case lengthened considerably. The diffusion resistance may be maintained over a correspondingly long period of time and the functionality of the coated component is ensured over this period of time;

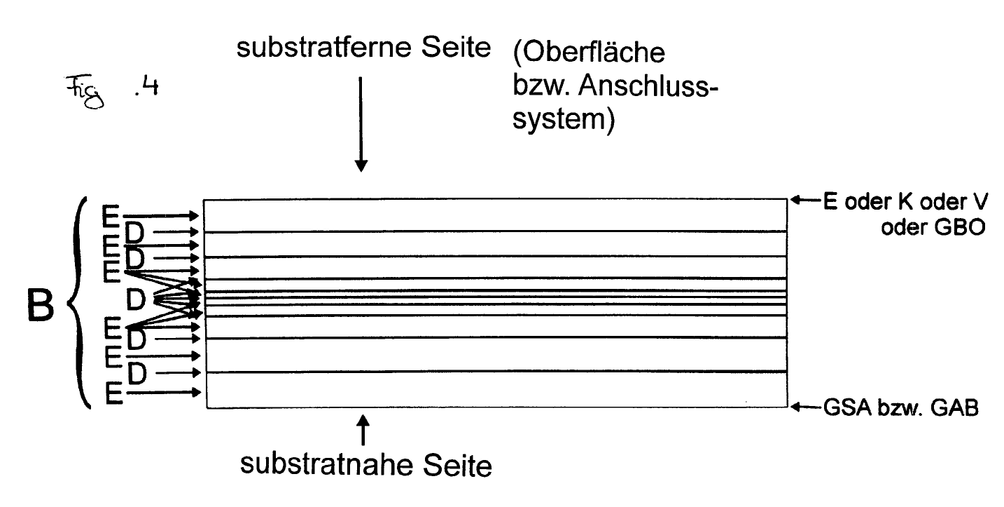

FIG. 4 shows the adaptation of the layer thicknesses of the resilient matrix layers E for maximum protection of the diffusion-resistant core layers D. The latter are embedded into the matrix layers at higher density (shorter distance) inside the layer system B. The thickness of the resilient matrix layers increases outwardly (toward the substrate side and toward the surface side);

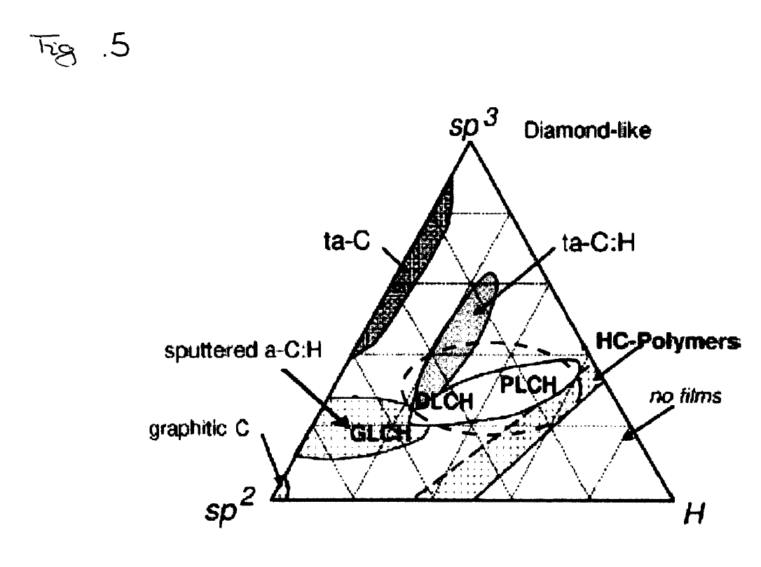

FIG. 5 is a ternary illustration of amorphous carbon layers, ordered in accordance with the bonding ratios and the hydrogen content in the layer according to John Robertson, Diamond-like amorphous carbon, Mat. Sci. Eng. R 37 (2002) 129; and

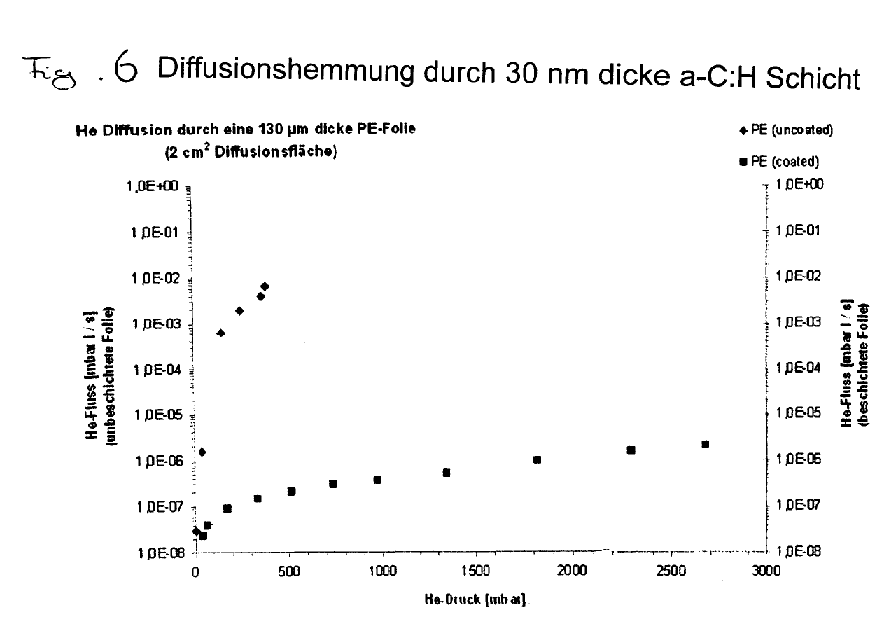

FIG. 6 shows the increase in the diffusion resistance of a 130 μm-thick polyethylene (PE) sheet by a 30 nm-thick amorphous carbon layer (diffusion-resistant core layer D1 (type 1)).

FIG. 1 shows the basic construction of the layer system. The layer system consists of thin, diffusion-resistant layers, known as “diffusion-resistant core layers” which in FIG. 1 are denoted by D and are integrated into thicker, resilient, less dense layers, known as “resilient matrix layers”, which in FIG. 1 are denoted by E. The transitions between the individual layers run seamlessly. Within these transition layers, which as “gradient layers” are also denoted in FIG. 1 as GED and GDE respectively, the layer properties, such as for example element composition, bonding ratios, density, modulus of elasticity, change gradually.

The system shown in FIG. 1 has the following layer sequence:

### Substrate/GSA or GAB/

### E/GED/D/GDE/E/GED/D/GDE/E/GED/D/GDE/E/GED/D/GDE/E/GED/D/GDE/E/GE

D/D/GDE/E/K or V or GBO/substrate-remote side

FIG. 2 shows the applying of the multilayer system to a substrate. The entire layer system is plasma-deposited and is applied either directly to a substrate, which in FIG. 2 is denoted by S, or to a layer which is deposited beforehand on the substrate in the same process and in FIG. 2 is denoted by A.

The multilayer system can be applied to different substrates, such as for example polymers, metals, ceramics, oxides or nitrites. The substrate may also be in the form of various coatings on the aforementioned materials (for example other layers applied in PVD, CVD and plasma-technological methods (for example metallic, carbidic, oxidic, nitridic and ceramic layers), various paints (for example polyurethane PU) and polymer coatings (such as for example parylene) and polymer layers deposited using plasma methods.

Exemplary Embodiment 1 made up of rf plasma-deposited, water-containing, amorphous carbon layers of various composition and layer properties:

| Layer | | | Layer |
|---|---|---|---|
| type | Material | Stoichiometry | thickness |
| Surface | | | |
| K and V | a-CH | 20% H | 40 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| D | a-CH | 35% H | 20 nm |
| | | content | |
| E | a-CH | 40% H | 30 nm |
| | | content | |
| GSB | Gradient PLCH (close to the | | 25 |
| | substrate) on a-C:H (toward E) | | |
| Substrate | | | |

Exemplary Embodiment 1 ensures the diffusion resistance, described in the present document, on the substrates parylene, polyethylene (PE), polyurethane (PU) and polypropylene (PP). The gradient transition layers GED and GDE form the transition between the a-C:H layers of various stoichiometry and have layer thicknesses of about 7 nm. Are not listed in the table. Exemplary Embodiment 1, described in the present document and applied to a 130 μm-thick PE sheet, reduces, for example, the diffusion of helium through the sheet from 4.5×10⁻³ mbar l/(s cm²) to a flow of 1×10⁻⁹ mbar l/(s cm²) at a helium overpressure of 400 mbar. The layer system, described in Exemplary Embodiment 1, without a substrate has a breakdown voltage of 260 V. The layer system described in Exemplary Embodiment 1 reduces the ion flow in an isotonic saline solution to currents below 1 pA/cm² at temperatures between 0 and 70° C.

Exemplary Embodiment 2 made up of rf plasma-deposited, water-containing, amorphous carbon layers of various composition and layer properties and also plasma-polymerised layers

| Layer | | | Layer | |
|---|---|---|---|---|
| type | Material | Stoichiometry | thickness | |
| Surface | | | | |
| K and V | a-CH | 20% H content | 40 | nm |
| E | a-CH | 40% H content | 30 | nm |
| D | a-CH | 35% H content | 20 | nm |
| E | a-CH | 40% H content | 30 | nm |
| D | a-CH | 35% H content | 20 | nm |
| E | a-CH | 40% H content | 30 | nm |
| D | a-CH | 35% H content | 20 | nm |
| E | a-CH | 40% H content | 30 | nm |
| D | a-CH | 35% H content | 20 | nm |
| E | a-CH | 40% H content | 30 | nm |
| D | a-CH | 35% H content | 20 | nm |
| E | a-CH | 40% H content | 30 | nm |
| GAB | Gradient PLCH (close to | | 25 | |
| | the substrate) on a-C:H | | | |
| | (toward E) | | | |
| A | Plasma-polymerised layer | According to H. Yasuda, T. Hsu, | 500 | nm |
| | | Surface Science 76 | | |
| | | (1978) 232 | | |
| GSA | Reactive plasma | Chemically modified | 10 | nm |
| | treatment with oxygen | substrate material | | |
| Substrate | | | | |

Exemplary Embodiment 2 ensures the diffusion resistance, described in the present document, on the substrates parylene, polyethylene (PE), polyurethane (PU) and polypropylene (PP). The gradient transition layers GED and GDE form the transition between the a-C:H layers of various stoichiometry and have layer thicknesses of about 7 nm. Are not listed in the table. Exemplary Embodiment 2, described in the present document and applied to a 130 μm-thick PE sheet, reduces, for example, the diffusion of helium through the sheet from 4.5×10⁻³ mbar l/(s cm²) to a flow of 1×10⁻⁹ mbar l/(s cm²) at a helium overpressure of 400 mbar. The layer system, described in Exemplary Embodiment 2, without a substrate has a breakdown voltage of 350 V. The layer system described in Exemplary Embodiment 2 reduces the ion flow in an isotonic saline solution to currents below 1 pA/cm² at temperatures between 0 and 70° C.

Suitable deposition conditions at the start of the process ensure a permanent connection of the layer system to the base (substrate). For this purpose, the transition layer denoted in FIG. 2 by GSA or by GSB is applied in the region bordering the substrate (GSA: gradient transition layer between the substrate and layer system A, GSB: gradient transition layer between the substrate and layer system B). The permanent connection of the layer system to the substrate is ensured, depending on the type of substrate used, in two ways. On the one hand, partial blending of both materials is attained by introducing layer atoms and/or layer molecules below the surface of the substrate. For this purpose, the deposition conditions are selected in such a way that ions of average energy (for example between 20 and 200 eV, in particular between 40 and 100 eV and specifically between 50 and 75 eV) infiltrate top layers of the substrate. The multifunctional multilayer system then grows out of this composite transition layer. On the other hand, the breaking-open of saturated bonds existing at the substrate surface provides at the surface of the substrate free bonds to which molecules and/or ions of the growing layer material can covalently bond. Reactive plasmas (such as for example oxygen plasmas, carbon dioxide plasmas) and/or ion bombardment (for example with noble gas ions, for example argon) and/or treatment with nitrogen plasma and/or mixtures of reactive plasmas (for example oxygen and/or carbon dioxide)) and noble gas plasmas (for example helium, argon) are, for example, used for breaking open the bonds. The multifunctional layer system is, as a result of this process step, securely anchored to the substrate via covalent bonds.

The layer system is provided on the side remote from the substrate with what is known as a capping layer. Four solutions, which are customised to the respective use of the layer, are proposed for the capping layer. These capping layers are denoted in FIG. 1 by E, K, V and GBO respectively. The capping layer is either the resilient matrix layer E, which has already been used in the layer system B, or an additionally applied, body-compatible layer K (if a layer system B is selected in which E does not have sufficient body compatibility). Solution 3 is an additionally applied protective layer V which provides protection from mechanical wear or chemical attacks. The fourth solution is the transition layer GBO which ensures permanent bonding of a further layer (or a layer system or an adhesive or a solid), for example by providing bond centres (for example of functional groups, such as for example amino groups —NH₂, and other nitrogen-containing and/or oxygen-containing functional groups, such as for example carboxyl groups —COOH, hydroxy groups —OH) adapted to the connection system and/or foreign atoms (for example silver) and also generally anchor points for forming carbonyl and/or ester and/or ether bonds.

The term “diffusion resistance” refers to the complete prevention or an extreme time delaying of the transportation of particles through the layer system.

Concentration gradients are generally the cause of such transportation of particles. In order to ensure diffusion resistance to gases, liquids, macromolecules, solid particles and ions, the layer system contains diffusion-resistant “core layers” of sufficient density which, as a result of high crosslinking, prevents infiltration of the aforementioned constituents.

The transportation, for example, of gases through a solid proceeds via two mechanisms. A diffusive flow via a solubility/diffusion mechanism is primarily observed in plastics materials. As modern analysis methods demonstrate, even compact, pore-free plastics materials are, in contrast to metals, more or less permeable to gases, vapours and liquids. The molecules which are in contact with the plastics materials are first absorbed at the surface, are dissolved with the plastics material, migrate, owing to the concentration gradient, slowly through the material (migration) and desorb again from the surface. In most other solids, flow caused by material defects predominates. Inhomogeneities, which allow the passage of atoms and even relatively large molecules, may be present in the form of microscopic cracks, channels or interconnected cavities (porosity).

In principle, numerous materials are suitable for forming diffusion-resistant layers. In practice, what matters is whether microcavities and, above all, continuous channels and cracks may be avoided during growth. Previous studies focus on the application of low-melting metals, transparent oxides or nitrides and hydrogenated carbon.

Metallised plastics materials have been used with great success since the 1970s in the packaging of food products. Aluminium layers can be economically manufactured in high-speed metallisers. With layer thicknesses of 20 nm, the diffusion of 12 μm-thick PET sheets may be improved by a factor of 100.

Transparent diffusion barrier layers use for this purpose silicon oxide, and some also use silicon nitride or combinations of both. These materials also allow a reduction of gas permeability by at least a factor of four, but usually by more than one order of magnitude.

The studies with hydrogenated carbon layers, which are promoted above all owing to the biocompatibility of this material, report reduction factors of between 4 and 20. In 200 nm-thick polymer films on 200 μm-thick membranes made of perfluorosulphonate ionomer (brand name Nafion from the company Du Pont), which were deposited in a low-pressure microwave discharge by decomposition of C₆H₁₄ and H₂, yielded, as the best value for the diffusion inhibition of methanol, a factor of 13 (M. Walker, K.-H. Baumgärtner, J. Feichtinger, M. Kaiser, E. Räuchle, J. Kerres, Surf. Coat. Techn. 116-119 (1999) 996). These sheets are used in fuel cells. They are intended to allow the diffusion of oxygen ions but prevent the passage of the fuel.

The studies with the decomposition of acetylene in a glow discharge produced the result that the softest layers (thickness of 362 nm), which were deposited on 12 μm-thick PET sheets (polyethylene terephthalate, brand name Hostaphan from Hoechst Diafoil) at the lowest bias voltage of 60 volts, display, at a factor of 20, the most marked diffusion inhibition. This may be interpreted to be the result of the fact that microcracks occur on more intensive compacting of the layers—an assumption which is also supported by the AFM observation. The fact that the weakening factors for all the gases examined (H₂, N₂, O₂, CO₂) are identical even in the best layer proves that molecular flow caused by defects still predominates even in this case. In the case of diffusion via the solubility mechanism, the permeability would, on the other hand, have to depend markedly on the chemical properties of the gases.

Observations of the diffusion of chemically inert gases (for example of helium gas) through the thin layers therefore provide information concerning the density of defects (pinholes, imperfections) in the layer.

It will be described hereinafter how, by combining various layer properties, the diffusion resistance of the multilayer system is maintained under various loads. In order to ensure diffusion resistance under chemical loading, for example a chemically aggressive environment (such as for example acids, lyes, electrolyte solutions, humid air), the layer system is either made up entirely of chemically inert compounds or provided with at least one chemically inert capping layer on the side remote from the substrate. In particular, the layer system is configured in such a way that partial swelling-up of the layer side remote from the substrate does not impair the diffusion resistance of the overall system.

In order to ensure diffusion resistance under thermal and/or mechanical loading, thin, diffusion-resistant “core layers” (D), made for example of amorphous carbon and/or oxides and/or nitrides and/or borides, are integrated into a flexible multilayered layer system consisting of resilient layers (E) based on carbon compounds, in particular on amorphous carbons and/or on plasma-polymerised compounds.

Forces acting on a layer system of this type, such as for example deformation caused by external pressure (for example expansion, compression) or temperature elevation, are distributed in the layer system. In this case, energy is deposited in the layer system as a whole. The layer regions which are more easily deformable (those are regions having a low modulus of elasticity) absorb more energy than those which are less easily deformable (i.e. regions having a higher modulus of elasticity). Neat arrangement of layer types of various moduli of elasticity leads, in the layer system proposed in the present document, to energy being discharged into the resilient matrix layers E. The diffusion-resistant core layers D are thus protected from deformation and the formation of microcracks. Without specifying the gradient layers (GDE and GED), FIG. 3a-c shows the layer sequence

side close to the substrate/GSA or GAB/E/D/E/D/E/D/E/D/E/D/E/D/E/side remote from the substrate.

As shown in FIGS. 3a to 3c, the diffusion resistance of the overall layer system is preserved despite the deformation thereof. This is supported by mathematical simulations for the crack formation of thin layers on polymer substrates.

Of course, the mechanism for the discharge of deformation energy described in the present document is also subject to limits. In the event of excessively intensive expansion, the energy is also deposited in the diffusion-resistant core layers D and microcracks, which influence the diffusion resistance, may be produced. In order to increase the tolerance of the layer system as a whole to deformations, a plurality of diffusion-resistant core layers D are therefore arranged in alternation with the resilient matrix layers E. In the event of intensive expansion of the layer system, the microcracks which are formed are statistically distributed over the entire layer surface. Particles diffusing through the layer system (molecules, atoms, ions) must pursue their path along the diffusion-resistant core layers D until they reach a crack. In this way, the path of the particles to be held off by the layer system is lengthened considerably and the diffusion resistance of the layer system is maintained over a correspondingly long period of time.

The number n of sequences of diffusion-resistant core layers/resilient matrix layers, including the transition layers (GDE and GED) listed in FIG. 1, is adapted to the respective application.

Likewise, for protecting the diffusion-resistant core layers D, the resilient matrix layers are deposited at variable layer thicknesses—adapted to the respective layer system (including the substrate and any subsequent layers). The layer thicknesses are larger on the substrate side and become smaller and smaller toward the centre of the layer system B in order then to become larger again on the surface side. A layer system of this type is shown in FIG. 4.

Without specifying the gradient layers (GDE and GED), FIG. 4 shows the layer sequence

side close to the substrate/GSA or GAB/E/D/E/D/E/D/E/D/E/D/E/D/E/D/E/D/E/D/E/supplementing K/V or
GBO/side remote from the substrate.

As a result, the diffusion-resistant core layers are arranged with higher density at the centre of the layer system. In the event of loading (for example deformation caused by bending), the outer layer regions are exposed to more intensive deformations. Depending on the moduli of elasticity of the individual layer types, there exists in the layer system what is known as a neutral plane which does not experience any deformation on loading of the overall system. Diffusion-resistant matrix layers D, which are positioned in proximity to this neutral plane, are exposed during deformation to less strong loads than those positioned further from the neutral plane—for example in proximity to one of the faces bordering the substrate or the surface. In a pure two-component system (layer S₁ having a thickness d₁ and a modulus of elasticity Y₁, layer S₂ having a thickness d₂ and a modulus of elasticity Y₂), the position of the neutral plane is precisely between the two components when Y₁×d₁²=Y₂×d₂². Layers having a lower modulus of elasticity must therefore be thicker than those having a higher modulus. If Y₁=40 GPa and Y₂=4 GPa, then it must be the case that d₂=3.16×d₁. These considerations are transferred accordingly to the multilayer system in order to ensure an advantageous position of most diffusion-resistant core layers.

The term “mechanical loading” includes, for example, the bending caused by movements of the substrate, for example as a result of the fact that the underlying substrate is a movable component, for example as a result of perfect that the substrate is a movable, oscillatory membrane, for example as a result of the fact that the substrate is a movable sheet which is exposed, for example, to a pressure gradient between two chambers or is used as part of a pump, for example as a result of the fact that the sheet is moved and deformed by mechanical forces in order to move a medium (for example a liquid) through a system.

The term “thermal loading” includes, for example, thermally induced deformations, which may—owing to different coefficients of thermal expansion—differ, (for example the change in length) of the base and/or of the layer system and/or of further layers, lying above the layer system, and/or solids.

The transition layers GSA and GSB respectively or the layer A and the transition layer GAB offer additional protection during thermal loading, as their composition is—if possible—selected in such a way that they have coefficients of thermal expansion in the order of magnitude of the substrate used in each case. The transition layers GSA and GSB respectively, proposed in the present document, have coefficients of thermal expansion of between 10 and 150 ppm/K, preferably between 15 and 100 ppm/K, in particular between 15 and 70 ppm/K, based in each case on the volume.

The multilayer system, proposed in the present document, on a substrate, which in FIG. 2 is denoted by B or, in systems comprising more than one multilayer system, by AB, tolerates expansions of at least 4%, in particular expansions of at least 10% and most particularly expansions of at least 25% without losing its diffusion resistance.

In order to ensure electrical insulation, nonconductors having high electrical resistance are predominantly used in the layer system. The electrical resistance of the layer system is at least 10¹⁸ Ωcm, preferably at least 10¹⁴ Ωcm and in particular 10¹⁷ Ωcm.

In order to ensure diffusion resistance under electrical loading (application of a voltage gradient), the layer system is, in particular, adapted for the transportation of ions and other charge carriers in an adjoining electric field. For this purpose, the layer system is chemically inert and consists predominantly of unipolar bonds and displays the arrangement described hereinbefore of the individual components. The multifunctional layer system proposed in the present document restricts the diffusion of ions in an aqueous solution even on application of an electric field gradient of at least 10⁴ V/cm, preferably of at least 10⁵V/cm and in particular of at least 10⁷ V/cm, the currents measured under the specified loads being less than 1 pA.

In order to provide protection from voltage breakdowns, care must be taken to ensure sufficient overall thickness of the layer system, on the one hand by depositing an insulating layer A under the layer B or, if A is dispensed with, by way of corresponding thickness of the layer B.

The breakdown voltage of the layer system proposed in the present document (AB or B in FIG. 2) is at least 2×10⁶ V/cm, preferably at least 4×10⁶ V/cm and in particular at least 10⁷V/cm.

In order to ensure body compatibility, the layer system is either constructed entirely on the basis of body-compatible layers, such as for example amorphous carbon layers (a-C:H, ta-C:H, DLC, DLCH, PLCH) or amorphous carbon layers containing foreign atoms (for example N and/or Si and/or Ag and/or F and/or O) or amorphous carbon layers comprising functional groups (such as for example —NH2, —NH, —COOH), or is capped by a layer of this type on the side facing the body. The biocompatibility of plasma-deposited carbon layers has already been demonstrated in numerous studies with various cell types (for example osteoblasts, fibroblasts, keratinocytes) and body fluids (in particular blood).

The coated substrate or component is protected from damage by UV radiation by the UV filter effect either of the capping layer or of the layer system as a whole. The layer system proposed in the present document filters out radiation having wavelengths of less than 400 nm.

Various layer types are used in the layer system proposed in the present document (B with GSB and K or V or GBO, or AB with GSA and GAB and K or V or GBO). Details concerning their composition and the bonding ratios present in the layer types are listed in Table 1.

Examples of the diffusion-resistant core layers (D) used include amorphous carbon layers, a-C:H and/or ta-C:H and/or ta-C and/or DLCH and/or AlOₓ and/or SiOₓ and/or ZrOₓ and/or TaOₓ and/or TiOₓ.

Examples of the resilient matrix layers (E) used include a-C:H and/or PLCH and/or HC polymers and/or plasma-polymerised layers.

The insulating layer A, which in certain application lies under the layer system B, consists, for example, of a-C:H and/or DLCH and/or PLCH and/or HC polymers and/or a plasma-polymerised layer. a-C:H and/or DLCH and/or PLCH are used as the capping layer K. a-C:H and/or ta-C:H and/or ta-C and/or DLCH are used as the capping layer V.

Amorphous carbon layers provided with functional groups and/or foreign atoms are used as the capping layer GBO.

Table 1 shows the element composition for various layer types.

### Table: Element Compositions for Different Layer Types:

TABLE 1
Stoichiometric composition of the layer types used in the layer system,
and also the bonding ratios present therein. The methods for producing the layer
types are also listed.

| Layer | Element | | | Production |
|---|---|---|---|---|
| types | composition | Stoichiometry | Bond types | method |
| D | AlOₓ | 0.6 < x < 1.5 | Oxidic | Magnetron |
| D | SiOₓ | 0.6 < x < 1.4 | | sputtering |
| D | ZrOₓ | 0.75 < x < 1.5 | | method |
| D | TaOₓ | 0.8 < x < 2.5 | | |
| D | TiOₓ | 0.75 < x < 1.25 | | |
| D | AlNₓ | 0.75 < x < 1.25 | Nitridic | |
| D | ZrNₓ | 0.75 < x < 1.25 | | |
| D | SiNₓ | 0.75 < x < 1.25 | | |
| D | BN | 0.75 < x < 1.25 | | |
| D | WC | 1.25 < x < 1.75 | Carbidic | |
| D | SiC | 0.25 < x < 1.25 | | |
| D | TiC | 1.25 < x < 1.75 | | |
| D | AlOₓ | 0.6 < x < 1.5 | Oxidic | |
| D | SiOₓ | 0.6 < x < 1.4 | | |
| D | ZrOₓ | 0.75 < x < 1.5 | | |
| D | TaOₓ | 0.8 < x < 2.1 | | |
| D | TiOₓ | 0.75 < x < 1.25 | | |
| D | a-C:H/DLCH | 20-40 at % H | Up to 60% sp³ | PECVD, |
| | diamond-like a- | | bonds high C—C | ECWR, ICP, |
| | C:H | | sp³ bond content | ECR |
| D | ta-C:H | 25-30 at % H | Up to 70% sp³ | PECVD, |
| | | | bonds at the same | ECWR, ICP, |
| | | | H content as or a | ECR |
| | | | lower H content | |
| | | | than DLCH | |
| D, K, V | a-C | See John Robertson: Mat. Sci. Eng. R | | Magnetron |
| | | 37 (2002) 129 | | sputtering, |
| D, K, V | ta-C | | | arc method |
| D, K, V | a-C:H: N/ | | | PECVD, |
| | DLCHN | | | ECWR, ICP, |
| D, K, V | ta-C:H:N | <=20 at % N | sp² cluster | ECR |
| | | | formation | |
| D, K, V | a-C:N | See John Robertson: Mat. Sci. Eng. R | | Magnetron |
| | | 37 (2002) 129 | | sputtering, |
| D, K, V | ta-C:N | | | arc method |
| E, K, A | a-C:H/PLCH, | >40 at % H, in | Up to 60% sp³ | PECVD, |
| | polymer-like a- | particular 40-50 | bonds; mostly C—H | ECWR, ICP, |
| | C:H a-C:H (soft) | at % H | sp³ bonds | ECR |
| E, K | a-C:H/GLCH, | <20 at % H | High sp² bond | |
| | graphite-like a- | | content, sp² | |
| | C:H | | cluster formation | |
| E, K, A | a-C:H: N/ | See John Robertson: Mat. Sci. Eng. R | | |
| | PLCHN | 37 (2002) 129 | | |
| E, K | a-C:H/ | | | |
| | polyacetylene | | | |
| E, A | Plasma- | | | |
| | polymerised | | | |
| | layers | | | |
| GBO | a-C:H—NH₂ | Density of the | Functional groups | |
| GBO | a-C:H—COOH | functional groups: | covalently bonded | |
| GBO | a-C:H—NH | 2.5/nm² to 10/nm² | to the surface | |
| GBO | a-C:H—OH | | | |

The number n of diffusion-resistant core layers (D) is at least 1 and is preferably between 2 and 200 and in particular between 3 and 9. The number of resilient matrix layers E is n+1. The thickness of the diffusion-resistant core layers (D) is between 3 and 150 nm, preferably between 3 and 75 nm and in particular between 3 and 30 nm. The diffusion-resistant core layers may be distributed in the resilient matrix layers equidistantly or randomly or else with increasing density toward the centre of the layer system B. The thickness of the resilient matrix layers (E) is between 10 and 300 nm, preferably between 10 and 200 nm and in particular between 10 and 100 nm. The resilient matrix layers E are between 2 and 100 times, preferably between 3 and 70 times and in particular at least (Y_{D}/Y_{E})^{0.5} as thick as the diffusion-resistant core layers D. With d_{bottom} and Y_{bottom} as the thickness or the modulus of elasticity of the overall system below the layer range having a high density of diffusion-resistant core layers and dₜₒₚ and Yₜₒₚ as the thickness or the modulus of elasticity of the overall system above the layer range having a high density of diffusion-resistant core layers, it is possible to select, for additional protection, the thickness dₜₒₚ in such a way that it is in the order of magnitude of (Y_{bottom}/Yₜₒₚ)^{0.5}×d_{bottom}. (d_{bottom}≈(Y_{bottom}/Yₜₒₚ)^{0.5}×d_{bottom}).

The transitions between the layers of different diffusion resistance and elasticity (GED, GDE) are between 0.5 and 30 nm, preferably between 0.5 and 20 nm and in particular between 0.5 and 10 nm. The thickness of the layer A is between 300 nm and 5 μm, preferably between 500 nm and 3 μm and in particular between 1 and 2 μm. The thickness of the transition layers GSA and GSB is between 0.5 and 200 nm, preferably between 0.5 and 50 nm and in particular between 0.5 and 20 nm.

The thickness of the capping layers E, K and V respectively is between 10 and 300 nm, preferably between 10 and 200 nm and in particular between 10 and 100 nm. The thickness of the capping layer GBO is between 0.5 and 30 nm, preferably between 0.5 and 20 nm and in particular between 0.5 and 10 nm.

In the CVD and PVD methods, which are in principle suitable for applying thin layers having thicknesses in the order of magnitude of up to a few μm, the material grows as a result of the accumulation of atomic or small molecular modules. In order to avoid cavities, and thus to suppress or restrict diffusion processes in the long term, it is necessary to arrange the layer modules (for example atoms, molecules, ions) as close together as possible during growth. If these particles cling to the point at which they arrive by chance, they form a relatively open structure. If, on the other hand, their movability is sufficient to find a site having as many bonding partners as possible, before their capacity for movement is restricted by the subsequently arriving atoms, this produces a compact network which allows hardly any diffusion.

The movability of the atoms at the surface is rendered possible mainly by thermal movement. The ratio of the growth temperature T_{A} to the melt temperature T_{S} is, in this case, highly relevant to the issue of compacting. As plastics materials are generally destroyed even at temperatures well below 100° C., it is not allowed to intensively heat the substrates in order to increase movability. Nevertheless, movability can also be significantly increased by ion bombardment. Plasma-physical methods (such as for example PECVF—plasma enhanced chemical vapour deposition, ECWR—electron cyclotron wave resonance, ECR—electron cyclotron resonance, magnetron sputtering rf glow discharge, DBD—dielectric barrier discharge, ICP—inductively coupled plasma, arc methods, FCVA—filtered cathodic vacuum arc, remote plasma deposition) are therefore used for deposition of the diffusion-resistant layer system proposed in the present document as, owing to the diffusion inhibition which is striven for, high density of the layer materials of the diffusion-resistant core layers D must be attained. Only once the growing layers have been intensively bombarded with ions of average energy is it ensured that microcavities are reliably avoided. As the ion bombardment is associated with an input of energy, only moderate growth rates may be striven for in the coating of plastics materials. In addition, it is advantageous if the ion energy is not much higher than is required for triggering the necessary site exchange processes. As the bonding energies are in the order of magnitude of a few eV, ion energies of a few 10 eV are optimal. This energy range would also appear to be particularly suitable for other reasons. At the low ion energy, the ions cannot infiltrate the pre-existing network. The internal stresses, which might lead to cracks, are substantially avoided. In addition, the layers growing under these conditions remain sufficiently resilient to be able to compensate for small mechanical stresses. The proposed layer system is produced in a single coating process. Time-intensive equipment changes and repeated surface preparations are thus dispensed with. Seamless transitions from the diffusion-resistant core layers to the resilient matrix layers are attained by gradually changing the process parameters (for example process gas or precursor flow, pressure, rf power, magnetic field strength, DC bias, target material, cathode voltage).

An example of a layer system based on carbon compounds will be given hereinafter.

Among the plasma-deposited layers, the amorphous carbons are distinguished by a particularly broad range of properties which are readily adjustable as a function of the deposition conditions. High hardness, a low coefficient of friction, optical transparency and good body compatibility are the best known properties of these materials. The electrically insulating layers, which are for the most part chemically inert, are made up of low molecular weight carbon compounds which are arranged with a certain short-range order in an otherwise unordered amorphous matrix. The amorphous carbon layers are combined to form the different groups listed in FIG. 5 in accordance with the bonding ratios of the carbon atoms. For the application desired in this case, the relevant layers are the hydrogen-containing a-C:H layers (ellipse with a red dotted line). They may display somewhat polymer-like (PLCH) or diamond-like (DLCH) properties. In the case of the carbon-based system described in the present document, the diffusion-resistant “core layers” consist of very thin, extremely dense carbon layers (for example a-C:H, DLC, DLCH). They are deposited in alternation with thicker, resilient and less dense carbon layers (for example a-C:H, PLCH, HC polymers). Owing to their chemical inertness and the non-polarity of the carbon/hydrogen bonds, the amorphous carbon-based, diffusion-resistant core layers (D) already effectively suppress diffusion as a consequence of solubility. For this purpose, the network has to be intensively crosslinked by a relatively high density of C—C bonds. This almost completely prevents the movement of foreign atoms, ions or molecules in the layer. In addition, the deposition conditions are selected in such a way that no cracks or other possibilities for passage, for example in the form of microscopic channels (pinholes), are formed during the layer growth.

As a result of applying between 3 and 150 nm, preferably between 3 and 75 nm and in particular between 3 and 30 nm-thick amorphous carbon layers, which in the layer system according to FIG. 1 presented in the present document are denoted as diffusion-resistant core layers D, to thin polymer sheets, the gas permeability thereof is reduced drastically by several orders of magnitude.

FIG. 6 shows the flow of helium through a 30 nm-thick, diffusion-resistant core layer D made of amorphous carbon (applied to a 130 μm-thick polyethylene sheet). In the case of an unloaded system (i.e. a system not exposed to expansion of length by heat or mechanical stress), a single, diffusion-resistant core layer allows a significant reduction of the helium flow to be attained in relation to the uncoated sheet. FIG. 6 shows the flow of helium through coated and uncoated, 130 μm-thick polyethylene (PE) sheets as a function of helium pressure. The average flow values measured for the coated sheets are denoted by squares (PE coated), those for the uncoated sheets are denoted by diamonds (PE uncoated). On the high-pressure side of the sheet, the helium pressure is increased until it reaches 3 bar or until a helium flow of 1×10⁻² mbar l/s is reached on the low-pressure side. At an overpressure of 400 mbar, the uncoated sheet is about 40,000 times more permeable to He gas than that which is coated with a 30 nm-thick, diffusion-resistant core layer D made of amorphous carbon.

In the layer system constructed on the basis of amorphous carbon layers, these diffusion-resistant core layers (D) are integrated into resilient matrix layers (E) which likewise consist of carbon compounds. The elasticity of carbon layers is generally determined by their content of C—C-sp³ bonds; their density depends substantially on the C concentration. The resilient matrix layers are distinguished by lower density and a low modulus of elasticity. The modulus of elasticity of a broad range of layers generally drops as the degree of crosslinking of the layer-forming molecules decreases. High crosslinking is attained in that ionised precursor molecules having a specific energy strike the surface, where they break open and divide the energy between the daughter atoms. In the case of CₘHₙ⁺ ions, the densest (highly crosslinked) layers are obtained at ion energies corresponding to 100 eV per C atom. At lower energies (per C atom), the deposited layer material is less dense and softer. A relatively resilient structure is therefore attained by lowering the ion energies (by way of the process parameters bias or pressure) or by using higher molecular weight organic precursors (for example para-xylene) at simultaneously low rf powers. That is to say, in order to prevent dissociation of the molecules into smaller fragments, a low Yasuda factor Y=W/FM (with W fed power in W, F monomer mass flow in sccm and M molecular weight of the monomer) is required. In the case of high molecular weight precursors, what are known as the plasma-polymerised layers are then obtained, plasma polymerisation generally representing the deposition of high molecular weight compounds (high molecular weight products) in electrical discharges which do not include amorphous carbon layers—even those described as being polymer-like.

However, the resilient matrix layers can also be deposited from low molecular weight hydrocarbons. The deposition conditions are in this case selected in such a way as to produce predominantly sp³-bonded, amorphous carbon layers having a high hydrogen content (40-60 atom %), most sp³ bonds of the C atoms leading to hydrogen atoms. These bonded H atoms have an inhibitory effect on the sp² clusters which are otherwise formed in amorphous carbon layers (olefin chains or aromatic rings), thus producing a weakly crosslinked “polymer-like” amorphous carbon network (PLCH). That takes place, for example, as a result of the fact that only low-energy ions (up to 20 eV per C atom) contribute to the formation of layers and the proportion of ions in the layer-forming particle flow is between 5% and 25%. In addition, the deposition rates are kept low to allow relaxation processes to take place. An (optionally additional) enlargement of the angles of incidence (measured toward the surface solder) of the plasma flow onto the surface also leads to layers having lower moduli of elasticity. An oblique particle flow or particle flow indirectly striking the substrate is preferable in the deposition of resilient layers.

Furthermore, a corresponding doping of the amorphous carbon layers with suitable foreign atoms reduces their modulus of elasticity. In the layer system proposed in the present document, oxygen, fluorine and vanadium are introduced into the resilient matrix layers.

The values for the modulus of elasticity of the resilient matrix layers E are between 1 and 70 GPa, preferably between 3 and 50 GPa and in particular between 3 and 20 GPa.

The layer system described in the present document based on carbon compounds already displays all the properties of a biocompatible layer. The layer of transition to the surface therefore does not have to be additionally made biocompatible.

Specific adaptations provide the layer system—beyond its elasticity and diffusion resistance—also with the possibility of coupling active substances to its surface. In particular, a charge with functional amino groups of at least 2.5/nm², preferably of at least 5/nm² and in particular of at least 10/nm² is attained using ammonia as a process gas.

The layer system described in the present document is deposited, for example, using a method coming under the class of the PECVD (plasma enhanced chemical vapour deposition) methods. An inductively coupled high-frequency plasma jet source is used to excite the process gases and precursors used.

Examples of the process gases used include hydrocarbon-containing gases, such as for example methane, acetylene, used individually or mixed with one another and/or mixed with nitrogen-containing process gases (such as for example nitrogen, ammonia) and/or mixed with fluorine-containing process gases (such as for example tetrafluorocarbon). Additionally vaporised liquid precursors (such as for example allylamine), organic and/or organometallic precursors (such as for example para-xylene, HMDSO) are also used.

The deposition method selected in the present document allows the properties of the layer-forming particle flow (ion energy and ion flow density) to be varied independently of one another, by way of a large number of process parameters (such as for example pressure, precursor gas flow and rf power), in such a way that amorphous carbon layers having quite different properties are deposited. At low process pressures (in the range of from 1×10⁻⁴ mbar-9×10⁻³ mbar), the residence time of the gas molecules in the source is reduced to a minimum, so that predominantly ionised and/or singly dissociated precursor gas particles (for example C₂H₂⁺, C₂H⁺ in the case of acetylene precursor gas) are produced. The particle flow thus produced is directed, under suitable geometry (direct or indirect or oblique incidence or diffuse or partly directed secondary plasma), onto the substrate, so that the deposition rates, and thus also the input of energy and the increase in temperature associated therewith of the substrate, remain limited. The carbon-based, diffusion-resistant core layers D are deposited from predominantly hydrocarbon-containing, gaseous precursors (for example acetylene) at a process gas pressure of between 3.0×10⁻⁴ mbar and 3.0×10⁻³ mbar, preferably between 5.0×10⁻⁴ mbar and 2.0×10⁻³ mbar and in particular between 7.0×10⁻⁴ mbar and 1.0×10⁻³ mbar. The rf power fed into the plasma is between 70 and 350 W, preferably between 100 and 300 W and in particular between 200 and 250 W. The geometrical arrangement is selected in such a way that the deposition rates are between 0.5 and 100 nm/min, preferably between 5 and 50 nm/min and in particular between 10 and 20 nm/min. The proportion of ions in the layer-forming particle flow is between 5 and 70%, preferably between 5 and 40% and in particular between 10 and 20%. The ion energies are between 8 and 100 eV/C-atom, preferably between 10 and 30 eV/C-atom and in particular between 10 and 20 eV/C-atom.

The carbon-based, resilient matrix layers E are deposited from predominantly hydrocarbon-containing, gaseous precursors—which may be obtained by vaporising—(for example methane and/or acetylene) at a process gas pressure of between 8.0×10⁻⁴ mbar and 9.0×10⁻³ mbar, preferably between 2.0×10⁻³ mbar and 7.0×10⁻³ mbar and in particular between 3.0×10⁻⁴ mbar and 6.0×10⁻³ mbar. The rf power fed into the plasma is between 50 and 350 W, preferably between 70 and 200 W and in particular between 100 and 175 W. The geometrical arrangement is selected in such a way that the deposition rates are between 0.5 and 100 nm/min, preferably between 5 and 50 nm/min and in particular between 10 and 20 nm/min. The proportion of ions in the layer-forming particle flow is between 5 and 25%, preferably between 5 and 20% and in particular between 5 and 15%. The ion energies are between 8 and 50 eV/C-atom, preferably between 8 and 30 eV/C-atom and in particular between 8 and 15 eV/C-atom. In the case of a metallic substrate, small contents of silicon (Si) are, for example, introduced into the amorphous carbon layer in order to ensure optimum adhesion to the base.

In the case of a polymer substrate, there is first applied, as layer A or layer E, which is close to the substrate, of the layer system B, a PLCH layer which has a coefficient of thermal expansion which is as adapted as possible and high elasticity and merges, as a result of gradual alteration of the process parameters, with a tight diffusion barrier layer (DLCH).

The region bordering any subsequent polymer layers displays, again, polymer-like properties and provides at the surface functional groups or reactive centres (for example —NH₂, —NH, —COON or N and/or Si and/or Ag and/or F and/or O) for effective bonding to the following layer.

If no further adjoining layer is applied, then the amorphous carbon-based layer system already displays good biocompatibility. If protection from wear is to be attained, then a DLCH layer is applied as the capping layer. For coupling of any active substances required for the respective application or for attaining a different functionality (for example for setting a specific wettability or free surface energy or for setting specific, for example bacteria-repellent or cell growth-promoting, properties), the amorphous carbon surface is equipped with the functional groups and bond centres listed in the present document in that, in the last step, the corresponding precursor gases—which may be obtained by vaporising—(for example ammonia, tetrachlorocarbon, organometallic precursors) are supplied or, as a result of cosputtering of solid targets, the desired elements are integrated. The transitions between the individual types of amorphous carbon layers ensure that—as described above—, in the diffusion barrier layer, movements of the workpiece or thermally induced expansion of the material surrounding the layer does not lead to the formation of cracks which would cause permeability in relation to the species to be held off (ions, gases, liquid molecules, particles). The materials are adapted to one another specifically with regard to the modulus of elasticity and coefficient of thermal expansion.

Oxidic layers are produced predominantly with the aid of sputtering processes. During the sputtering process (also cathode atomisation), gas ions are accelerated from a plasma onto a target. The target material is in this case atomised and the (sputtered) material which is struck free is subsequently deposited on a substrate located in proximity to the target. The sputtering is carried out in a closed recipient which, before the start of the deposition, is pumped off to a vacuum which is as good as possible. In order to ignite a plasma, a process gas is introduced up to a typical working pressure of between 10⁻³ and 5×10⁻² mbar. The target is set to an electrical potential (DC or rf). As a result of natural cosmic radiation, there are at all times a few ions and electrons which are accelerated in the electric field and lead, as a result of impacts in the gas, to collective ionisation. The plasma which is produced in this case is compacted by suitable magnetic fields in such a way that the greatest intensity prevails directly on the target surface.

The resulting layer properties may be determined in a purposeful manner by way of the plasma parameters such as the electron energy distribution function, electron density and plasma potential in different gas compositions. For the production of oxidic layers, a gas mixture of Ar (60% to 90%) and O₂ (40% to 10%) is introduced into the process chamber. Preferably, an AC voltage having a frequency of 13.6 MHz is applied to the metallic sputtering target. A low plasma power (100 W to 150 W) is set so that low process temperatures are attained. The materials Al, Si, Zr, Ta and Ti are used as the sputtering target. The stoichiometry of the layer is determined by way of the Ar/O₂ gas ratios, the sputtering power and the distance from the substrate to the ion source.

For depositing the oxide-based multilayer system proposed in the present document, the described sputtering methods are combined with the PECVD method described hereinbefore in the layer system. The, for example oxide-based, layer system contains oxidic, diffusion-resistant core layers which are embedded, for example, in resilient matrix layers deposited using the PECVD method. The resilient matrix layers are in this case both the resilient matrix layers described hereinbefore and polymer-like, plasma-deposited layers deposited from vaporised organometallic precursors. In the case of, for example, SiOₓ as the diffusion-resistant core layers, HMDSO is expedient as a corresponding organometallic precursor. The diffusion-resistant SiOₓ layer may also be deposited from the organometallic precursor using the PECVD method, oxygen gas being added to set the appropriate stoichiometry (Table 1), a method which facilitates the deposition of the gradual transition layers GEB and GBE. In the case, for example, of TaOₓ as the diffusion-resistant core layers, the organometallic precursor is expediently tantalum tetraethoxydimethylaminoethoxide (TAT DMAE) or tantalum ethylate (Ta(C₂H_{S}O)₅) or, in the case of ZrOₓ, zirconium acetate which can accordingly be used with oxygen gas in the PECVD method. The transition from oxidic to polymer-like layers is controlled, on use of organometallic precursors (MO), for example by way of the flow ratio (oxygen/MO) and the power which is fed into the plasma and ensures a corresponding dissociation of the MO molecules. The diffusion-resistant core layers D are deposited at a high oxygen/MO ratio and high power; the resilient matrix layers are deposited from the organometallic precursor at low power (low Yasuda factor). The statements made hereinbefore also apply accordingly to nitride and carbide-based layer systems. In these cases, oxygen, as the precursor gases, is replaced by nitrogen-containing or hydrocarbon-containing precursor gases.

The plasma-deposited, electrically insulating, diffusion-resistant, resilient, body-compatible layer system proposed in the present document may, on account of its multifunctional properties, be used in a large number of areas of application. In principle, all applications are possible in which a mass flow has to be suppressed. Examples include packaging sheets and containers, in particular in the food sector for food products and beverages, in the pharmaceutical industry for, for example, medicaments and in the chemical industry for, for example, readily volatile solvents, petrols, corrosive liquids, hygroscopic solids and powders. Examples include tanks for fuels such as, for example, petrol, hydrocarbons and hydrogen. The layer system may be introduced in seals and covers and significantly improve the barrier function thereof. As a result of its high tolerance to deformations, it may also be used in components subjected to extreme loads as a result of mechanical pressure, such as for example O-rings, and also offer additional diffusion protection for the purposes of sealing. Likewise, it imparts new functionality to fabrics and items of clothing. The layer system imparts to plastics materials a resilient barrier layer which prior to gas emission provides protection from harmful additives, such as for example plasticisers and solvents, and imparts additional UV protection to the plastics material. This lengthens the service life of the plastics materials and extends their field of use to biological and medical areas. The layer system protects flat screens (what are known as FPDs, flat panel displays, such as for example liquid crystal displays, LCDs) and also organic light emitting diodes (OLEDs) from diffusion and rapid ageing processes and is suitable for the encapsulation of what are known as MEMSs (microelectromechanical systems) such as are found, for example, in printer heads of inkjet printers or in acceleration sensors for triggering airbags. The layer system serves to encapsulate electrodes and electronic circuits in order to protect them, in particular, from the infiltration of moisture in environments having high air humidity, in aqueous solutions or other solvents. In particular, the layer system protects electrodes and electronic circuits from ion diffusion and prevents voltage breakdowns. The additional outstanding body compatibility of the layer system makes it particularly suitable for the encapsulation of medical implants in that it, on the one hand, keeps body fluids, ions, proteins and other molecules away from the interior of the implant and, on the other hand, prevents a release of (possibly harmful) implant constituents into the surrounding tissue. Thus, body compatibility and functionality of the implant are ensured even under the above-mentioned loads. The residence time of the implant in the body may thus be substantially lengthened. The layer system is particularly suitable for the encapsulation of electrically active medical implants. These include, for example, implants for functional electrical stimulation (FES), implants with electrode systems for detecting bioelectric potential differences, for detecting neuronal innervation patterns, implants for electrical stimulation of individual nerve fibres, overall precisely what are known as neuroprostheses, pacemakers, implants for dynamic myoplasty, implants for diaphragmatic or phrenic nerve stimulation and for encapsulating electromechanical implants (such as for example artificial hearts, VAD (ventricular-assisted device) systems, total artificial heart systems) and for encapsulating what are known as BioMEMSs (microimplants based on micromechanical systems).

1. A multilayer system on a substrate, the multilayer system being applied to the substrate with the aid of plasma deposition, wherein the multilayer system is constructed in such a way that it has substantial diffusion resistance to ions in an aqueous solution, the current produced by diffusion of ions being less than 6.5×10⁻⁸ A/cm² when an electric field gradient of more than 10⁴ V/m is applied.

2. The multilayer system according to claim 1, wherein the multilayer system has a water vapor flow rate of less than 3.7×10⁻⁸ mbar·L·s⁻¹/cm² and a gas flow rate of less than 1.5×10⁻⁷ mbar·L·s⁻¹/cm² whereby the multilayer system exhibits substantial diffusion resistance to gases and/or water vapor and/or solvent vapors.

3. The multilayer system according to claim 1, wherein the multilayer system is constructed in such a way that the multilayer system is chemically stable in relation to acids and lies in a pH range between 0 and 14.

4. The multilayer system according to claim 1, wherein substantial diffusion resistance is provided in the event of expansion of the multilayer system in any desired directions parallel to the substrate of less than 25%.

5. The multilayer system according to claim 1, wherein the multilayer system is constructed in such a way that the multilayer system is thermally stable in the range from −50° C. to 200° C.

6. The multilayer system according to claim 1, wherein the substantial diffusion resistance is provided in aqueous solutions and in bodily fluids.

7. A multilayer system on a substrate, the multilayer system comprising:
at least one core layer consisting of amorphous carbon layers, a-C:H, ta-C:H, ta-C, DLCH, AlOₓ, SiOₓ, ZrOₓ, TaOₓ, and/or TiOₓ;
at least one resilient matrix layer consisting of a-C:H, PLCH, HC polymers, and/or plasma-polymerized layers;
at least one layer promoting adhesion to the substrate;
a capping layer on the side remote from the substrate; and
transition gradient layers provided between the various layers.

8. The multilayer system according to claim 7, wherein the number of diffusion-resistant core layers is between 1 and 200.

9-14. (canceled)

15. The multilayer system according to claim 7, the transition gradient layers comprising first transition gradient layers between the resilient matrix layers and the core layers and second transition gradient layers between the core layers and the resilient matrix layers.

16. (canceled)

17. The multilayer system according to claim 15, the transition gradient layers further comprising third transition gradient layers between the multilayer system and the substrate, the third transition gradient layers having coefficients of thermal expansion of between 10 and 150 ppm/K in three dimensions.

18. (canceled)

19. The multilayer system according to claim 1 further comprising an insulating layer connected to the substrate.

20. The multilayer system according to claim 19, wherein the insulating layer consists of a-C:H, DLCH, PLCH, HC polymers, and/or a plasma polymerized layer.

21. The multilayer system according to claim 19 further comprising a substrate-remote capping layer comprising a-C:H, DLCH, ta-C:H, ta-C, and/or PLCH.

22. The multilayer system according to claim 21, wherein the substrate-remote surface of the substrate-remote capping layer has bond centers adapted to provide a connection system.

23. The multilayer system according to claim 22, wherein the bond centers comprise nitrogen- and/or oxygen-containing functional groups.

24. The multilayer system according to claim 23, wherein the functional groups comprise amino groups, carboxyl groups, hydroxy groups, and/or foreign atoms and the functional groups serve as anchor points for forming carbonyl, ester, and/or ether bonds.

25. The multilayer system according to claim 1, wherein the substrate is a polymer-like substrate.

26. The multilayer system according to claim 25, wherein the polymer-like substrate comprises parylene.

27. The multilayer system according to claim 26, further comprising a connection system comprising a polymer-like material, in particular parylene.

28. The multilayer system according to claim 1, wherein the multilayer system is embodied as a UV filter and the transmission of electromagnetic radiation having wavelengths of between 200 and 400 nm is less than 20%, the filter effect being independent of the substrate.

29. The multilayer system according to claim 1, wherein the multilayer system has high body compatibility.

30. The multilayer system comprising at least two multilayer systems according to claim 1, wherein a connection layer is arranged between the two multilayer systems.

31-49. (canceled)

50. Use of a multilayer system according to claim 1,
for encapsulating electrically active medical implants;
for implants for functional electrical stimulation (FES), for implants with electrode systems for detecting bioelectric potential differences, for detecting neuronal innervation patterns;
for implants for electrical stimulation of nerve fibers, in particular individual nerve fibers, neuroprostheses, pacemakers;
for implants for dynamic myoplasty, implants for diaphragmatic or phrenic nerve stimulation;
for encapsulating electromechanical implants (such as for example artificial hearts, ventricular-assisted device systems, total artificial heart systems);
for encapsulating what are known as BioMEMSs (micro-implants based on micro-electromechanical systems)
for encapsulating electrodes and electronic circuits in order to protect them, in particular, from the infiltration of moisture in environments having high air humidity, in aqueous solutions or other solvents;
for flat screens (what are known as FPDs, flat panel displays, such as for example liquid crystal displays, LCDs) and also organic light emitting diodes (OLEDs) in order to protect these from diffusion and rapid ageing processes;
for encapsulating what are known as MEMSs (micro-electromechanical systems), in particular in printer heads of inkjet printers, acceleration sensors for triggering airbags;
for suppressing diffusion of matter;
on packaging sheets and containers, in particular in the food sector for food products and beverages, in the pharmaceutical industry for medicaments and in the chemical industry for readily volatile solvents, petrols, corrosive liquids, hygroscopic solids and powders;
on tanks and storage containers for fuels such as, for example, petrol, hydrocarbons, hydrogen and highly volatile explosive mixtures;
on seals and covers for improving the barrier function, in particular on mechanically loaded components such as, in particular, O-rings; and
on fabrics and items of clothing.
